(19) United States
(12) Patent Application Publication
(10) Pub. No.: US 2025/0073684 A1
(43) Pub. Date: Mar. 6, 2025

(54) COPPER-ZINC ALLOY CATALYST, AND PREPARATION METHOD AND USE THEREOF

(71) Applicant: Taiyuan University of Technology, Taiyuan City (CN)

(72) Inventors: Wei Huang, Taiyuan City (CN); Enjuan Ma, Taiyuan City (CN); Qian Zhang, Taiyuan City (CN); Min Tian, Taiyuan City (CN)

(73) Assignee: Taiyuan University of Technology, Taiyuan City (CN)

(21) Appl. No.: 18/422,533

(22) Filed: Jan. 25, 2024

(30) Foreign Application Priority Data
Aug. 29, 2023 (CN) 202311091983X

(51) Int. Cl. B01J 23/80; B01J 35/61; B01J 35/64; B01J 37/06; B01J 37/34; B01J 38/50; C07C 27/06; C22C 1/08; B01J 35/63

(52) U.S. Cl. CPC B01J 23/80; B01J 35/612; B01J 35/647; B01J 37/06; B01J 37/343; B01J 38/50; C07C 27/06; C22C 1/08; B01J 35/633

(57) ABSTRACT

Provided are a copper-zinc alloy catalyst, and a preparation method and use thereof. The method for preparing the copper-zinc alloy catalyst includes: subjecting copper-zinc alloy particles to pretreatment to obtain the copper-zinc alloy catalyst; alternatively, subjecting copper-zinc alloy particles to pretreatment and partial dezincification in sequence to obtain the copper-zinc alloy catalyst.

### CROSS REFERENCE TO RELATED APPLICATION

This patent application claims the benefit and priority of Chinese Patent Application No. 202311091983X, entitled “COPPER-ZINC ALLOY CATALYST, AND PREPARATION METHOD AND USE THEREOF” filed with the China National Intellectual Property Administration on Aug. 29, 2023, the disclosure of which is incorporated by reference herein in its entirety as part of the present application.

### TECHNICAL FIELD

The present disclosure relates to the technical field of catalysts, and in particular to a copper-zinc alloy catalyst, and a preparation method and use thereof.

### BACKGROUND

Coal, petroleum, and natural gas are the three major energy systems with large consumption. However, none of these primary energy sources are renewable. At present, great efforts are being made to develop new energy systems, but coal may still be a main energy source in the next 50 years. Carbon emissions from coal consumption account for almost 70% of all energy carbon emissions, which leads to a low coal utilization rate. Therefore, it is necessary to promote clean and efficient utilization of coal. It is an effective method for clean and efficient utilization of coal to synthesize a low-carbon alcohol from a synthesis gas produced by coal gasification.

The low-carbon alcohol refers to C₁₋₆ mixed alcohols, which are generally used as an additive for fuel or cleaning gasoline. This additive mainly promotes clean combustion of fuel by increasing an oxygen content and an octane number of the fuel. A key for synthesizing the low-carbon alcohol from the synthesis gas is to develop catalysts with high selectivity for C₂₊ alcohols. There are currently four main categories of catalysts for synthesizing the low-carbon alcohol: Rh-based catalysts, Mo-based catalysts, modified F-T synthesis catalysts, and modified methanol synthesis catalysts. The Rh-based catalysts have mild reaction conditions and a high selectivity to the low-carbon alcohol, but are not conducive to industrialization since Rh is a noble metal with high cost. The Mo-based catalysts are resistant to sulfur and not prone to carbon deposition, but show harsh reaction conditions and could produce sulfides that are difficult to remove in the product. The modified F-T synthesis catalysts have a stronger carbon chain growth ability, but could generate products that are widely distributed and difficult to separate. The modified methanol synthesis catalysts mainly include a modified high-temperature Zn—Cr catalyst and a modified low-temperature Cu-based catalyst. The modified high-temperature Zn—Cr catalyst has relatively high reaction temperature and pressure, resulting in high energy consumption and difficulty in industrialization; and the modified low-temperature Cu-based catalyst has low cost and mild reaction conditions, making it easier to achieve industrialization, but shows a relatively low yield of the low-carbon alcohol.

So far, research on copper alloys as catalysts mainly focuses on Cu—Co bimetallic alloy catalysts. In the existing technology, Cu—Co bimetallic alloy is used as a catalyst, which requires a carrier and an additive (such as La₂O₃), and the carrier and additive interact and block with each other to promote dispersion of the Cu—Co alloy, thereby improving an activity of the catalyst to catalyze the synthesis gas to produce the low-carbon alcohol. In addition, Co is an F-T element and has strong carbon chain growth ability, making the product difficult to be separated, and the additive is a rare earth metal oxide, which is costly and not conducive to industrialization. In view of this, it has become an urgent technical problem in the field to provide a copper alloy catalyst that does not require any additives or carriers to achieve an excellent catalytic effect in producing the low-carbon alcohol from the synthesis gas.

### SUMMARY

An object of the present disclosure is to provide a copper-zinc alloy catalyst, and a preparation method and use thereof. In the present disclosure, the copper-zinc alloy catalyst could effectively catalyze a synthesis gas to produce a low-carbon alcohol by using only a copper-zinc alloy without adding any additives or carriers.

To achieve the above object, the present disclosure provides the following technical solutions:

The present disclosure provides a method for preparing a copper-zinc alloy catalyst, including:
subjecting copper-zinc alloy particles to pretreatment to obtain the copper-zinc alloy catalyst; alternatively,
subjecting copper-zinc alloy particles to pretreatment and partial dezincification in sequence to obtain the copper-zinc alloy catalyst.

In some embodiments, the copper-zinc alloy particles are prepared from brass.

In some embodiments, the pretreatment is performed by a process including: subjecting the copper-zinc alloy particles to ultrasonic cleaning by using an organic solvent, ultrasonic cleaning by using heated deionized water, and drying in sequence.

In some embodiments, the ultrasonic cleaning by using the organic solvent is conducted at a frequency of 15 KHz to 25 KHz.

In some embodiments, the ultrasonic cleaning by using heated deionized water is conducted at a frequency of 20 KHz to 35 KHz and at a temperature of 50° C. to 80° C.

In some embodiments, the partial dezincification is performed by a process including: mixing a resulting pretreated copper-zinc alloy with an acid solution to obtain a mixture; subjecting the mixture to replacement to obtain a reaction system; and subjecting the reaction system to washing with a weakly alkaline solution, washing with water, and drying in sequence.

In some embodiments, the acid solution is one selected from the group consisting of a nitric acid solution, a sulfuric acid solution, and a hydrochloric acid solution.

The present disclosure further provides a copper-zinc alloy catalyst prepared by the method described above.

The present disclosure further provides use of the copper-zinc alloy catalyst in preparation of a low-carbon alcohol from a synthesis gas, including:
subjecting the copper-zinc alloy catalyst to impurity removal to obtain a cleaned copper-zinc alloy, introducing the synthesis gas into the cleaned copper-zinc alloy, and subjecting the synthesis gas to reaction at a temperature of 240° C. to 340° C. to obtain the low-carbon alcohol; wherein the synthesis gas is introduced at a pressure of 3 MPa to 5 MPa.

In some embodiments, the synthesis gas is introduced at a flow rate of 80 mL/min to 120 mL/min and a volume space velocity of 9,600 h⁻¹ to 14,400 h⁻¹. In some embodiments, the synthesis gas consists of H₂ and CO, and a volume ratio of the H₂ to the CO is in a range of (1-3):1.

The present disclosure provides a method for preparing a copper-zinc alloy catalyst, including: subjecting copper-zinc alloy particles to pretreatment to obtain the copper-zinc alloy catalyst; alternatively, subjecting copper-zinc alloy particles to pretreatment and partial dezincification in sequence to obtain the copper-zinc alloy catalyst. In the present disclosure, the copper-zinc alloy particles are used as catalysts. A Cu—Zn alloy phase in the copper-zinc alloy serves as a catalytic active site to improve a catalytic performance of the copper-zinc alloy catalyst. The copper-zinc alloy is subjected to the partial dezincification, such that Zn is partially removed in the alloy, so as to expand a specific surface area of the copper-zinc alloy particles, thereby providing more active sites for catalyzing the synthesis of the low-carbon alcohol from the synthesis gas, and achieving a desirable catalytic effect without using a carrier or additive. The results of examples show that when being used alone, the copper-zinc alloy catalyst shows a CO conversion rate of 20.86% and a C₂₊ alcohol selectivity of 62.78%, showing excellent catalytic effect and C₂₊ alcohol selectivity.

### BRIEF DESCRIPTION OF THE DRAWINGS

FIG. 1 shows a scanning electron microscopy (SEM) image magnified 100 times of the copper-zinc alloy catalyst prepared in Example 1 of the present disclosure;

FIG. 2 shows an SEM image magnified 100 times of the copper-zinc alloy catalyst prepared in Comparative Example 1 of the present disclosure;

FIG. 3 shows an SEM image magnified 100 times of the copper-zinc alloy catalyst prepared in Comparative Example 2 of the present disclosure;

FIG. 4 shows an X-ray diffraction (XRD) pattern of the copper-zinc alloy catalysts prepared in Example 1 and Comparative Examples 1 to 2 of the present disclosure;

FIG. 5 shows an SEM image magnified 5,000 times of the copper-zinc alloy catalyst prepared in Example 1 of the present disclosure;

FIG. 6 shows an SEM image magnified 5,000 times of the copper-zinc alloy catalyst prepared in Example 2 of the present disclosure;

FIG. 7 shows an SEM image magnified 5,000 times of the copper-zinc alloy catalyst prepared in Example 3 of the present disclosure;

FIG. 8 shows an SEM image magnified 5,000 times of the copper-zinc alloy catalyst prepared in Example 4 of the present disclosure;

FIG. 9 shows an SEM image magnified 5,000 times of the copper-zinc alloy catalyst prepared in Example 5 of the present disclosure;

FIG. 10 shows an XRD pattern of the copper-zinc alloy catalysts prepared in Examples 1 to 5 of the present disclosure;

FIG. 11 shows a CO conversion rate of the copper-zinc alloy catalysts in Use Example 1 and Comparative Use Examples 1 to 2 of the present disclosure;

FIG. 12 shows a total alcohol selectivity of the copper-zinc alloy catalysts prepared in Use Example 1 and Comparative Use Examples 1 to 2 of the present disclosure; and

FIG. 13 shows a C₂₊ alcohol proportion of the copper-zinc alloy catalysts prepared in Use Example 1 and Comparative Use Examples 1 to 2 of the present disclosure.

### DETAILED DESCRIPTION OF THE EMBODIMENTS

The present disclosure provides a method for preparing a copper-zinc alloy catalyst, including: subjecting copper-zinc alloy particles to pretreatment to obtain the copper-zinc alloy catalyst; alternatively,
subjecting copper-zinc alloy particles to pretreatment and partial dezincification in sequence to obtain the copper-zinc alloy catalyst.

In one embodiment of the present disclosure, the copper-zinc alloy particles are subjected to the pretreatment to obtain the copper-zinc alloy catalyst.

In another embodiment of the present disclosure, the copper-zinc alloy particles are subjected to the pretreatment and partial dezincification in sequence to obtain the copper-zinc alloy catalyst.

In some embodiments of the present disclosure, the copper-zinc alloy particles are prepared from brass.

In the present disclosure, the brass is an industrial brass powder with only two elements: Cu and Zn. In the present disclosure, the copper-zinc alloy particles are limited to the above material, which could benefit a catalytic activity of the catalyst.

In some embodiments of the present disclosure, the pretreatment is performed as follows: subjecting the copper-zinc alloy particles to ultrasonic cleaning by using an organic solvent, ultrasonic cleaning by using heated deionized water, and drying in sequence.

In some embodiments of the present disclosure, the ultrasonic cleaning by using the organic solvent is performed by acetone ultrasonic cleaning and ethanol ultrasonic cleaning conducted in sequence. In some embodiments, the acetone ultrasonic cleaning is conducted twice, and the ethanol ultrasonic cleaning is conducted twice. In some embodiments, the ultrasonic cleaning by using the organic solvent is conducted at a frequency of 15 KHz to 25 KHz, and preferably 20 KHz.

In some embodiments of the present disclosure, the ultrasonic cleaning by using heated deionized water is conducted three times at a frequency of 20 KHz to 35 KHz, and preferably 30 KHz. In some embodiments, the ultrasonic cleaning by using heated deionized water is conducted at a temperature of 50° C. to 80° C., and preferably 60° C. There are no special limitations on operations of the ultrasonic cleaning by using the organic solvent and the ultrasonic cleaning by using heated deionized water, and conventional ultrasonic operations conducted by those skilled in the art may be used. In the present disclosure, the times and frequency of the ultrasonic cleaning are limited to the above ranges, which could ensure more thorough cleaning and impurities removal in the copper-zinc alloy particles.

In some embodiments of the present disclosure, the drying is conducted at a temperature of 90° C. to 120° C., and preferably 110° C. In some embodiments, the drying is conducted for 10 hours to 15 hours, and preferably 12 hours. In the present disclosure, the temperature and time for drying are limited to the above ranges, which could ensure sufficient drying of the pretreated copper-zinc alloy particles.

In some embodiments of the present disclosure, the partial dezincification is performed as follows: mixing a resulting pretreated copper-zinc alloy with an acid solution to obtain a mixture; subjecting the mixture to replacement to obtain a reaction system; and subjecting the reaction system to washing with a weakly alkaline solution, washing with water, and drying in sequence.

In some embodiments of the present disclosure, the acid solution is one selected from the group consisting of a nitric acid solution, a sulfuric acid solution, and a hydrochloric acid solution, and preferably the hydrochloric acid solution. In some embodiments, the hydrochloric acid solution is a concentrated hydrochloric acid with a mass fraction of 36% to 38%. In the present disclosure, the concentration of the acid solution is limited to the above ranges, which could facilitate the replacement of the copper-zinc alloy with the acid solution.

In some embodiments of the present disclosure, the replacement is conducted at ambient temperature for 1 hour to 36 hours, preferably 12 hours to 36 hours, and more preferably 20 hours to 28 hours. In the present disclosure, the temperature and time for replacement are limited to the above ranges, which could ensure that a part of the zinc in the alloy is removed, resulting in increased pores on a surface of the catalyst, thereby improving a catalytic performance of the catalyst.

In some embodiments of the present disclosure, shaking is conducted after a certain interval during the displacement, preferably 2 hours to 6 hours. In the present disclosure, air bubbles generated in the reaction could be quickly removed by shaking, such that the partial dezincification of the copper-zinc alloy could be achieved.

In some embodiments of the present disclosure, the weakly alkaline solution is a sodium carbonate solution or a potassium carbonate solution, and preferably the potassium carbonate solution. In some embodiments, the weakly alkaline solution has a molar concentration of 0.1 mol/L to 0.5 mol/L.

In the present disclosure, there are no special limitations on operations of the cleaning with a weakly alkaline solution and washing with water, and operations commonly used by those skilled in the art may be used. A resulting filtrate is titrated by using a silver nitrate solution to determine that there is no Cl⁻. In the present disclosure, other impurity ions on the surface of the copper-zinc alloy particles could be removed by cleaning and neutralizing a residual acid on the surface of the catalyst with the weakly alkaline solution, and then washing with water.

In some embodiments of the present disclosure, the drying is conducted at a temperature of 90° C. to 120° C., and preferably 110° C. There is no special limitation on a drying time, as long as the washed copper-zinc alloy catalyst could be dried.

In the present disclosure, the copper-zinc alloy particles are used as a catalyst. A Cu—Zn alloy phase in the copper-zinc alloy serves as a catalytic active site to improve a catalytic performance of the copper-zinc alloy catalyst. The copper-zinc alloy is subjected to partial dezincification, such that Zn is partially removed in the alloy, so as to expand a specific surface area of the copper-zinc alloy particles, thereby providing more active sites for catalyzing the synthesis of the low-carbon alcohol from the synthesis gas, and achieving a desirable catalytic effect without using a carrier or additive.

The present disclosure further provides a copper-zinc alloy catalyst prepared by the method described above.

The present disclosure further provides use of the copper-zinc alloy catalyst in preparation of a low-carbon alcohol from a synthesis gas, including the following steps:
subjecting the copper-zinc alloy catalyst to impurity removal to obtain a cleaned copper-zinc alloy, introducing the synthesis gas into the cleaned copper-zinc alloy, and subjecting the synthesis gas to reaction at a temperature of 240° C. to 340° C. to obtain the low-carbon alcohol; wherein the synthesis gas is introduced at a pressure of 3 MPa to 5 MPa.

In some embodiments of the present disclosure, a device for producing the low-carbon alcohol from the synthesis gas is a fixed bed reactor. In some embodiments, the copper-zinc alloy catalyst is loaded at a content of 0.5 g to 1.5 g, and preferably 1 g. In the present disclosure, the loading content of the catalyst are set within the above ranges, which could facilitate the reaction of the synthesis gas to the low-carbon alcohol.

In some embodiments of the present disclosure, the copper-zinc alloy catalyst is placed in a quartz reaction tube and then placed in the fixed bed reactor to conduct the reaction. In the present disclosure, the copper-zinc alloy catalyst is placed in the quartz reaction tube first, which could avoid an influence caused by direct contact of the catalyst with the inner wall of a stainless steel tube of the fixed bed reactor.

In some embodiments of the present disclosure, the impurity removal is conducted as follows: feeding N₂ into the quartz reaction tube and then heating the copper-zinc alloy catalyst to a temperature of 300° C. There are no special limitations on a N₂ flow rate, a heating rate, and an impurity removal time, and parameters commonly used by those skilled in the art could be used.

In some embodiments of the present disclosure, the synthesis gas is introduced at a flow rate of 80 mL/min to 120 mL/min, and preferably 100 mL/min. In some embodiments, the synthesis gas is introduced at a volume space velocity of 9,600 h⁻¹ to 14,400 h⁻¹, and preferably 12,000 h⁻¹. In some embodiments, the synthesis gas consists of H₂ and CO with a volume ratio of (1-3):1, and preferably 2:1. In the present disclosure, the flow rate, components, and ratio of the synthesis gas are limited to the above described ranges, which could make it possible to better synthesize the low-carbon alcohol.

In some embodiments of the present disclosure, the synthesis gas is introduced at a pressure of 3 MPa to 5 MPa, and preferably 4 MPa. In the present disclosure, the reaction is performed at a temperature of 240° C. to 340° C., and preferably 300° C. In some embodiments, the synthesis gas is heated to the temperature for reaction at a heating rate of 2° C./min. In the present disclosure, the pressure and temperature for the reaction are limited to the above described ranges, which could ensure a better progress of the reaction and improve a conversion rate of the synthesis gas.

In some embodiments of the present disclosure, after the reaction, a resulting reaction product is divided into a gas phase and a liquid phase through cyclic condensation, and the gas phase and the liquid phase are quantitatively analyzed by chromatography separately.

The embodiments of the present disclosure will be clearly and completely described below with reference to the examples of the present disclosure. Apparently, the described examples are merely some rather than all of the embodiments of the present disclosure. All other embodiments obtained by those skilled in the art based on the embodiments of the present disclosure without creative efforts shall fall within the scope of the present disclosure.

### Example 1

5 g of brass particles were added into a beaker, subjected to ultrasonic cleaning by using 25 mL of acetone twice at ambient temperature for 30 min each time, and subjected to ultrasonic cleaning by using absolute ethanol twice at an ultrasonic frequency of 20 KHz for 30 min each time, and then subjected to ultrasonic cleaning by using deionized water 3 times at 30 KHz in sequence to obtain cleaned brass particles. The cleaned brass particles were dried in an oven at 110° C. for 12 hours to obtain a copper-zinc alloy catalyst, recorded as Cu-ye.

### Comparative Example 1

Comparative Example 1 was performed according to Example 1 except that the brass particles were replaced with red copper particles to obtain a copper-zinc alloy catalyst, recorded as Cu-pu.

### Comparative Example 2

Comparative Example 2 was performed according to Example 1 except that the brass particles were replaced with bronze particles to obtain a copper-zinc alloy catalyst, recorded as Cu-cy.

### Example 2

The copper-zinc alloy catalyst obtained in Example 1 was placed into a small beaker containing 15 mL of concentrated hydrochloric acid (36% to 38%), and subjected to partial dezincification at room temperature for 12 hours, with shaking every 2 hours to remove air bubbles on a catalyst surface as soon as possible. After the reaction, the alloy was washed twice with prepared 0.2 mol/L sodium carbonate until neutral, and then washed with deionized water to remove excess impurity ions on the catalyst surface, and dried at 110° C. to obtain a copper-zinc alloy catalyst, recorded as Cu-ye-12 h.

### Example 3

Example 3 was performed according to Example 2 except that the partial dezincification was conducted for 20 hours, with shaking every 4 hours to obtain a copper-zinc alloy catalyst, recorded as Cu-ye-20 h.

### Example 4

Example 4 was performed according to Example 2 except that the partial dezincification was conducted for 28 hours, with shaking every 6 hours to obtain a copper-zinc alloy catalyst, recorded as Cu-ye-28 h.

### Example 5

Example 5 was performed according to Example 2 except that the partial dezincification was conducted for 36 hours, with shaking every 6 hours to obtain a copper-zinc alloy catalyst, recorded as Cu-ye-36 h.

### Use Example 1 and Comparative Use Examples 1 to 2

The copper-zinc alloy catalysts prepared in Example 1 and Comparative Examples 1 to 2 were used to produce a low-carbon alcohol from a synthesis gas by a fixed-bed reactor: the copper-zinc alloy catalyst was loaded into a quartz reaction tube at a loading content of 1 g, and heated at 2° C./min to 300° C. for 3 hours in a N₂ (100 mL/min) atmosphere to remove impurities. After cooling the copper-zinc alloy catalyst to ambient temperature, the synthesis gas was introduced instead of N₂ until the synthesis gas had a pressure of 4 MPa, and then subjected to a reaction at a temperature of 240° C. to 340° C. for 2 hours. The synthesis gas was heated to the reaction temperature at a heating rate of 2° C./min. The synthesis gas was consisted of H₂ and CO at a volume ratio of 2:1. The synthesis gas was introduced at a total gas flow of 100 mL/min, and a volume space velocity of 12,000 h⁻¹. A resulting reaction product was divided into a gas phase and a liquid phase by using cyclic condensation, and the gas phase and the liquid phase were quantitatively analyzed by chromatography separately.

### Use Examples 2 to 5

The copper-zinc alloy catalysts prepared in Examples 2 and 5 were used to produce a low-carbon alcohol from a synthesis gas by a fixed-bed reactor: the copper-zinc alloy catalyst was loaded into a quartz reaction tube at a loading content of 1 g, and heated at 2° C./min to 300° C. for 3 hours in a N₂ (100 mL/min) atmosphere to remove impurities. After cooling the copper-zinc alloy catalyst to ambient temperature, the synthesis gas was introduced instead of N₂ until the synthesis gas had a pressure of 4 MPa, and subjected to a reaction at a temperature of 300° C. for 2 hours. The synthesis gas was heated to the reaction temperature at a heating rate of 2° C./min. The synthesis gas was consisted of H₂ and CO at a volume ratio of 2:1. The synthesis gas was introduced at a total gas flow of 100 mL/min, and a volume space velocity of 12,000 h⁻¹. A resulting reaction product was divided into a gas phase and a liquid phase by using cyclic condensation, and the gas phase and the liquid phase were quantitatively analyzed by chromatography separately.

### Comparative Use Example 3

Comparative Use Example 3 was performed according to Use Example 2 except that the catalyst in Comparative Use Example 3 is prepared according to Chinese patent 201711158742.7, specifically, an electrolytic brass product disclosed in example 4 of the US201711158742.7 was used as a catalyst (i.e., Cat-4) to catalyze the synthesis gas to produce alcohol. The amount of the Cat-4 is 0.5 g. Before the reaction, the catalyst was reduced with a H₂/N₂ gas mixture at a volume ratio of 6:1 at 470° C. for 6 hours, and then the reaction was performed at a lowered temperature of 450° C.

In the present disclosure, an appearance of the copper-zinc alloy catalysts was observed by an electron microscope.

In the present disclosure, a physical phase of the copper-zinc alloy catalysts was characterized by an XRD diffractometer.

In the present disclosure, the resulting reaction products obtained from Use Examples of the catalyst to produce low-carbon alcohol from the synthesis gas were subjected to chromatographic quantitative analysis by a Haixin GC950 chromatography.

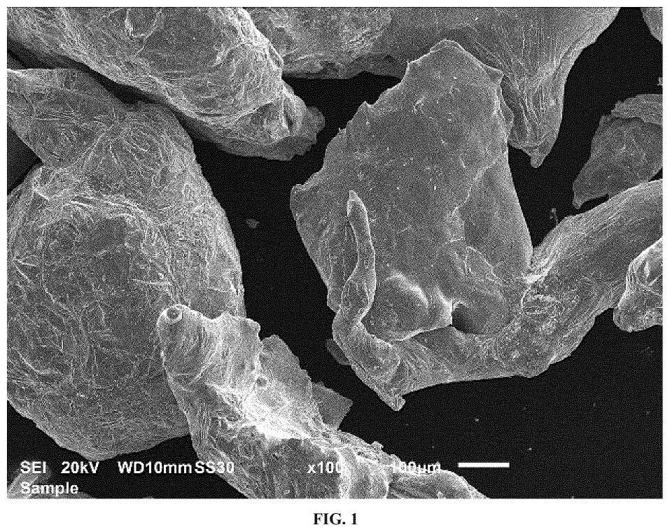

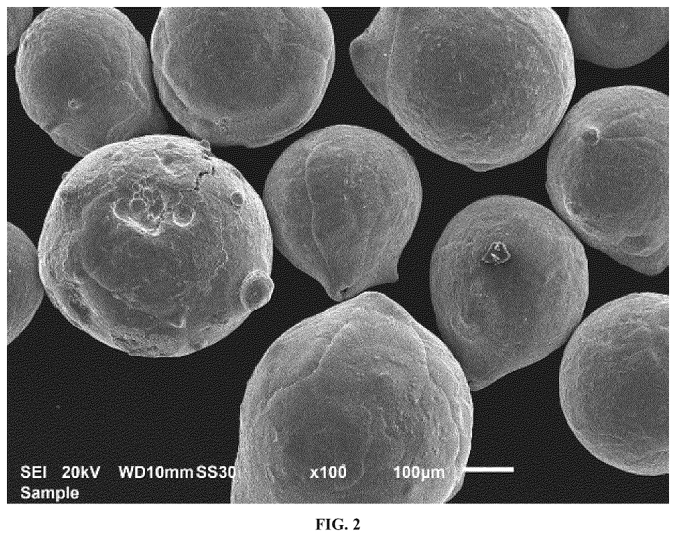

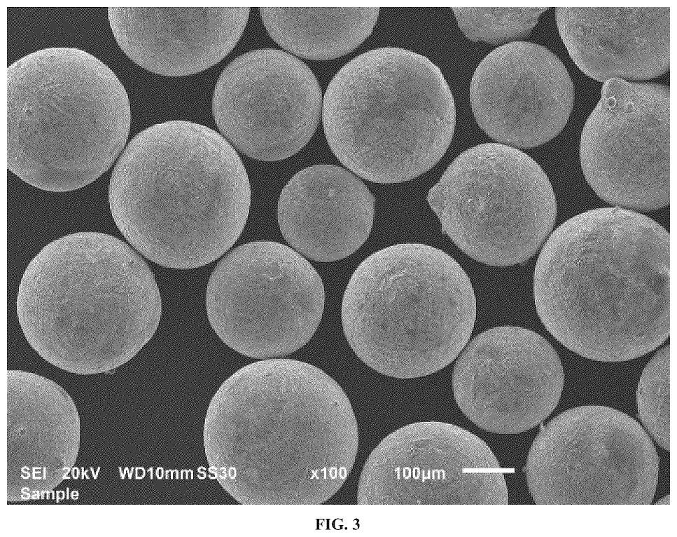

FIG. 1 to FIG. 3 show SEM images magnified 100 times of the copper-zinc alloy catalysts prepared in Example 1 and Comparative Examples 1 to 2. From FIG. 1 to FIG. 3, it can be seen that the Cu-ye catalyst has an irregular shape, and the Cu-pu and Cu-cy catalysts both have a relatively-smooth spherical shape.

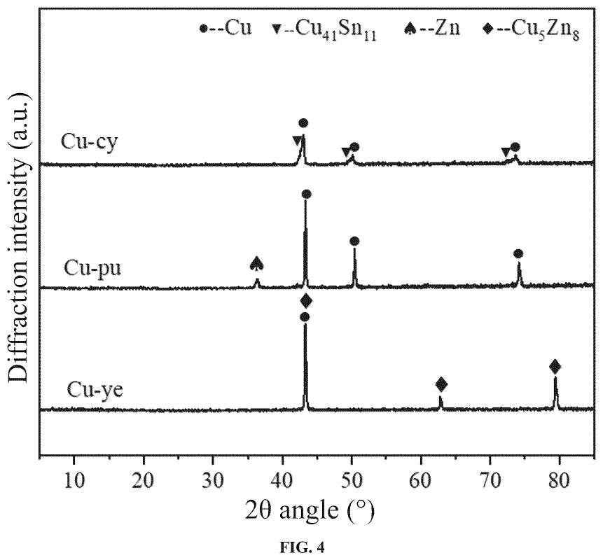

FIG. 4 shows XRD patterns of the copper-zinc alloy catalysts prepared in Example 1 and Comparative Examples 1 to 2. From FIG. 4, it can be seen that the Cu-ye catalyst only contains a Cu₅Zn₈ alloy phase, the Cu-pu catalyst shows a Cu phase and a Zn phase, and the Cu-cy catalyst contains a Cu phase and a Cu₄₁Sn₁₁ alloy phase.

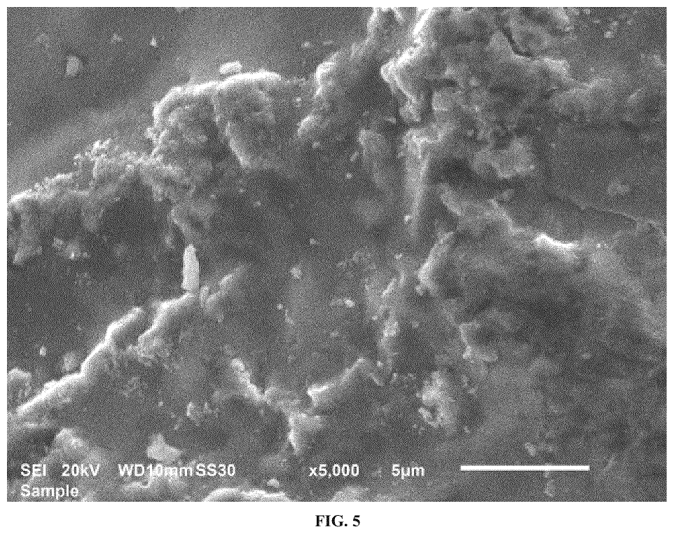

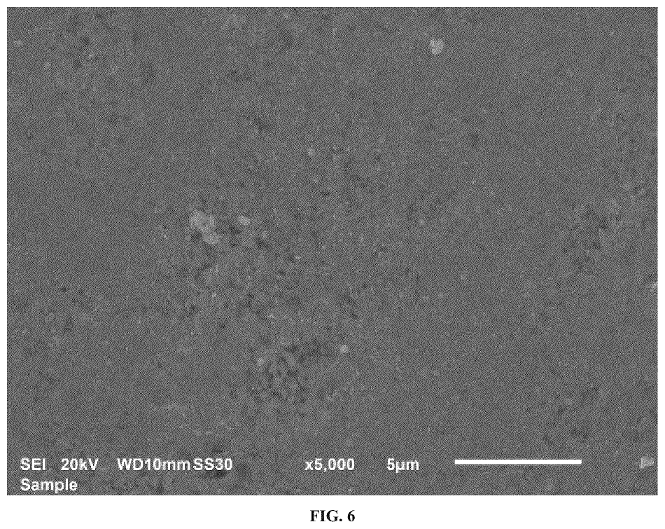

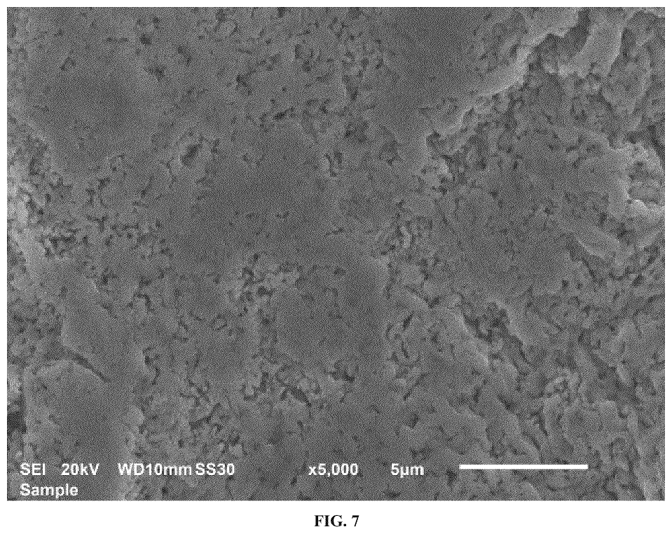

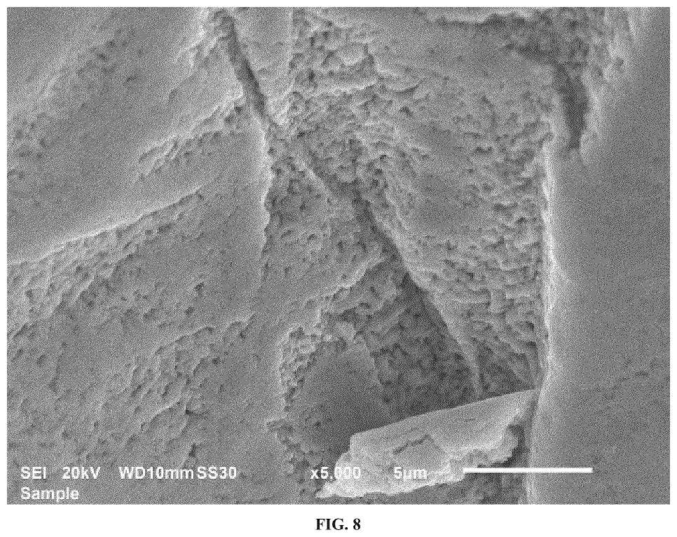

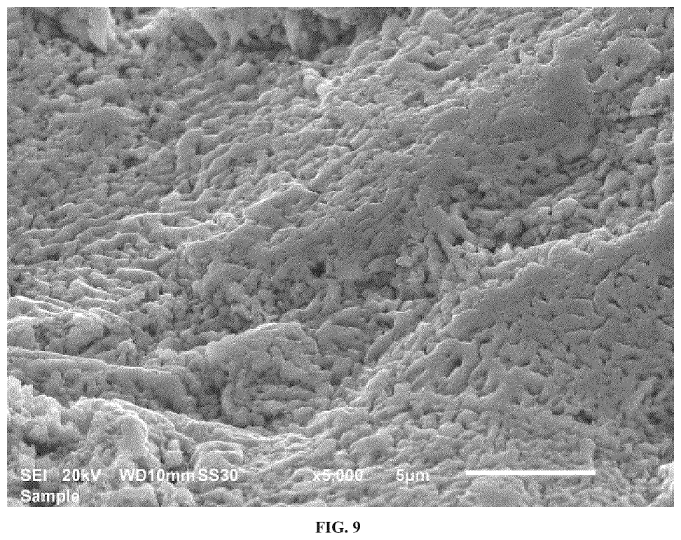

FIG. 5 to FIG. 9 show SEM images magnified 5,000 times of the copper-zinc alloy catalysts prepared in Examples 1 to 5. From FIG. 5 to FIG. 9, it can be seen that surface pores of the catalyst are increased after the dezincification.

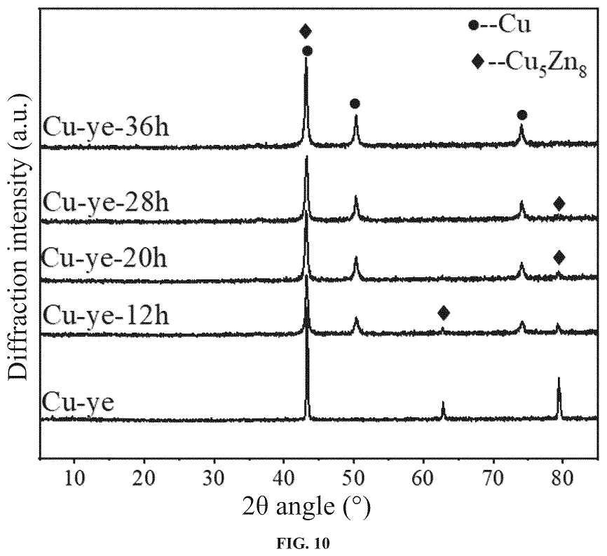

FIG. 10 shows XRD patterns of the copper-zinc alloy catalysts prepared in Examples 1 to 5. From FIG. 10, it can be seen that the Cu-ye catalyst without the dezincification only has the Cu₅Zn₈ alloy phase. During the reaction with hydrochloric acid, Zn is partially removed. As a removal time increased, a peak of the Cu₅Zn₈ alloy phase gradually decreased, and a peak of the Cu phase became sharper. After part of the Zn is removed, the Cu₅Zn₈ alloy and Cu coexist in the catalyst.

The copper-zinc alloy catalysts prepared in Examples 1 to 5 are tested for a specific surface area, a pore volume, and a pore size, and the test results are shown in Table 1.

TABLE 1
Specific surface area, pore volume, and pore size of the
copper-zinc alloy catalysts prepared in Examples 1 to 5

| | Specific surface area/ | Pore volume/ | Average pore size/ |
|---|---|---|---|
| Catalyst | (m² · g⁻¹) | (cm³ · g⁻¹) | nm |
| Cu-ye | 0.47 | 0.11 | 3.40 |
| Cu-ye-12 h | 0.79 | 0.18 | 15.29 |
| Cu-ye-20 h | 1.00 | 0.23 | 17.52 |
| Cu-ye-28 h | 1.21 | 0.28 | 21.12 |
| Cu-ye-36 h | 1.86 | 0.43 | 23.85 |

As shown in Table 1, after the partial dezincification of the copper-zinc alloy catalysts prepared in Examples 2 to 5, the specific surface area, pore volume, and pore size of the catalyst all increased.

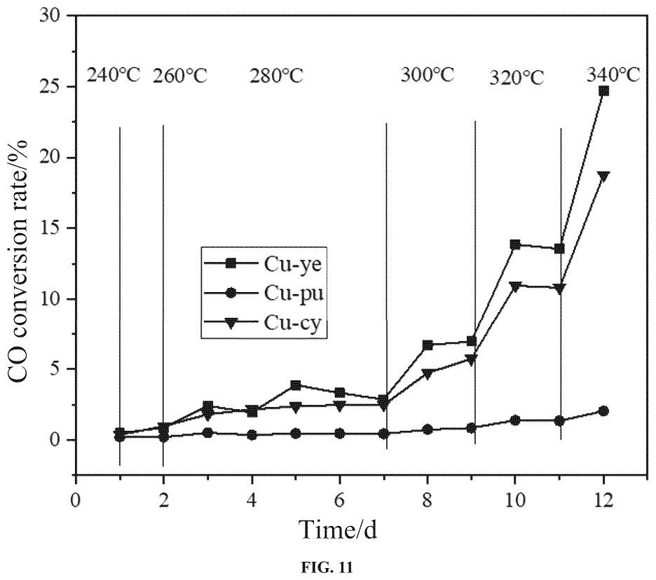

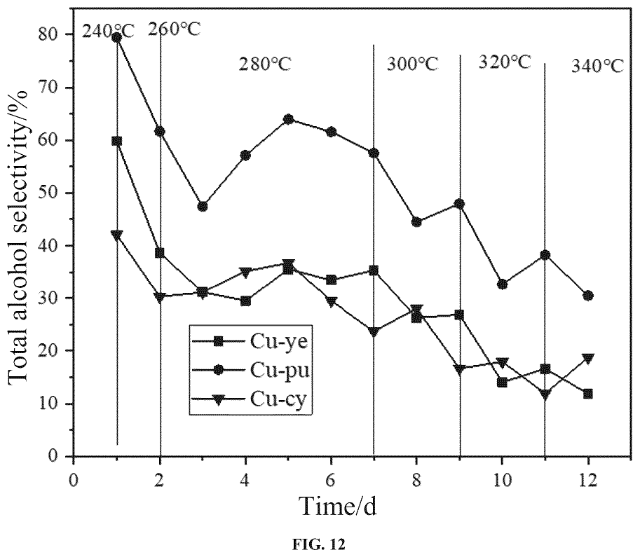

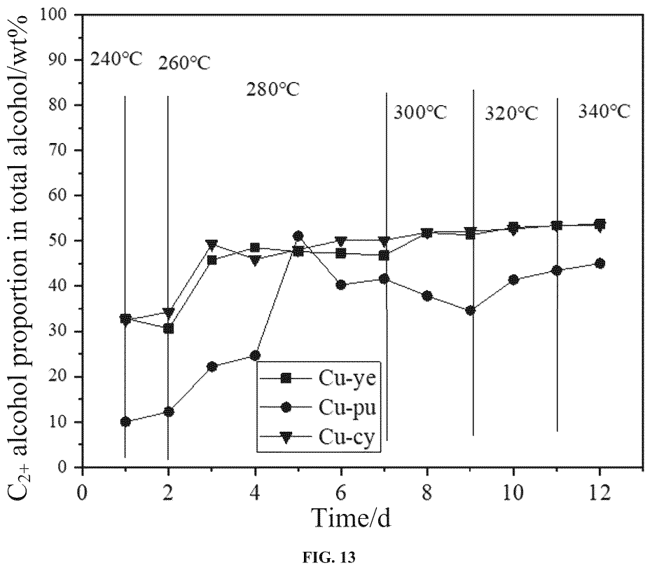

FIG. 11 to FIG. 13 show a CO conversion rate, a total alcohol selectivity, and a C₂₊ alcohol proportion of the copper-zinc alloy catalysts prepared in Use Example 1 and Comparative Use Examples 1 to 2. From FIG. 11 to FIG. 13, it can be seen that with the reaction temperature increased, the copper-zinc alloy catalysts have increased CO conversion rate and decreased total alcohol selectivity, and the C₂₊ alcohol proportion in the total alcohol is increased and gradually stabilized at around 54%.

The results of reaction activity evaluation at 300° C. of the copper-zinc alloy catalysts prepared in Use Example 1 and Comparative Use Examples 1 to 2 are shown in Table 2.

TABLE 2
Results of reaction activity evaluation at 300° C.
of the copper-zinc alloy catalysts prepared in Use
Example 1 and Comparative Use Examples 1 to 2

| | | | | | | Alcohol | | | |
|---|---|---|---|---|---|---|---|---|---|
| | | | | | | product | | | |
| | Con- | | | | | distribution/ | | Alcohol | |
| | version | Product selectivity/mol % | | | | wt % | | yield/g · mL⁻¹ · h⁻¹ | |
| Catalyst | rate/% | CHₓ | CO₂ | ROH | DME | CH₃OH | C₂₊OH | ROH | C₂₊OH |
| Cu-ye | 6.96 | 60.49 | 12.48 | 26.97 | 0.06 | 48.67 | 51.33 | 64.35 | 27.25 |
| Cu-pu | 0.85 | 40.79 | 10.76 | 47.97 | 0.48 | 65.43 | 34.57 | 1.60 | 0.45 |
| Cu-cy | 5.74 | 58.73 | 13.09 | 28.11 | 0.07 | 47.79 | 52.21 | 71.15 | 30.45 |

From Table 2, it can be seen that catalysts containing only Cu—Zn alloy could also be used to synthesize the low-carbon alcohol. Compared with the Cu-ye catalyst, the Cu-pu catalyst does not have an alloy phase, and shows the worst catalytic effect, a low CO conversion rate, and a low C₂₊ alcohol proportion. Although the Cu-cy catalyst contains a Cu—Sn alloy phase, the catalytic effect is also significantly reduced compared with the catalyst containing Cu—Zn alloy phase. Therefore, the copper-zinc alloy catalyst containing the Cu—Zn alloy phase shows a good catalytic effect.

The results of activity evaluation of Use Examples 1 to 5 and Comparative Use Example 3 are shown in Table 3.

TABLE 3
Results of activity evaluation of Use
Examples 1 to 5 and Comparative Use Example 3

| | Con- | Product | | | | Product | | Alcohol | |
|---|---|---|---|---|---|---|---|---|---|
| | version | selectivity/mol % | | | | distribution/wt % | | yield/g · mL⁻¹ · h⁻¹ | |
| Catalyst | rate/% | CHₓ | CO₂ | ROH | DME | CH₃OH | C₂₊OH | ROH | C₂₊OH |
| Cu-ye-12h | 18.89 | 46.57 | 19.06 | 34.37 | 0.00 | 40.17 | 59.83 | 261.28 | 156.32 |
| Cu-ye-20h | 18.57 | 54.31 | 20.96 | 24.73 | 0.00 | 57.49 | 42.51 | 177.12 | 75.27 |
| Cu-ye-28h | 20.05 | 47.38 | 21.75 | 30.87 | 0.00 | 39.34 | 60.66 | 218.62 | 132.62 |
| Cu-ye-36h | 20.86 | 47.94 | 21.58 | 30.48 | 0.00 | 37.22 | 62.78 | 200.60 | 125.94 |
| Cu-ye | 6.96 | 60.49 | 12.48 | 26.97 | 0.06 | 48.67 | 51.33 | 64.35 | 27.25 |
| Cat-4 | 41.20 | 42.80 | 15.60 | 41.60 | 0.00 | 74.30 | 25.70 | — | — |

Table 3 shows the results of activity evaluation of Use Examples 1 to 5 and Comparative Use Example 3. Compared with the catalyst (Cu-ye) in Use Example 1, the catalysts of Use Examples 2 to 5 show an increased conversion rate, an increased total alcohol selectivity, and an increased alcohol yield, indicating that the catalytic properties of the catalysts could be improved after the dezincification. In Comparative Use Example 3, the Cat-4 catalyst is used, where the Cat-4 catalyst is required to be reduced at a higher temperature for 6 hours (the Examples of the present disclosure are not required to be reduced, while Comparative Example 3 is reduced at 470° C.) and reacted at a higher temperature (the Examples of the present disclosure are reacted at 300° C., while Comparative Example 3 are reacted at 450° C.) to achieve a CO conversion rate of 41.2%. Moreover, the C₂₊ alcohol in the product of the Comparative Example 3 only accounts for 25.7%. Even if the reaction is conducted at 450° C., carbon chain growth effect is not desirable. On the contrary, the catalysts in the Examples of present disclosure have a better selectivity for C₂₊ alcohols when the reaction is conducted at 300° C., and the C₂₊ alcohol proportion is as high as 62.78%.

In the present disclosure, the copper-zinc alloy catalysts have a CO conversion rate of 20.86% and a C₂₊ alcohol selectivity of 62.78% when used alone, showing excellent catalytic effect and C₂₊ alcohol selectivity, which may have better industrialization prospects.

The above descriptions are merely preferred embodiments of the present disclosure. It should be noted that those skilled in the art may further make several improvements and modifications without departing from the principle of the present disclosure, but such improvements and modifications should be deemed as falling within the scope of the present disclosure.

What is claimed is:

1. A method for preparing a copper-zinc alloy catalyst, comprising:
subjecting copper-zinc alloy particles to pretreatment to obtain the copper-zinc alloy catalyst; alternatively,
subjecting copper-zinc alloy particles to pretreatment and partial dezincification in sequence to obtain the copper-zinc alloy catalyst.

2. The method of claim 1, wherein the copper-zinc alloy particles are prepared from brass.

3. The method of claim 1, wherein the pretreatment is performed by a process comprising: subjecting the copper-zinc alloy particles to ultrasonic cleaning by using an organic solvent, ultrasonic cleaning by using heated deionized water, and drying in sequence.

4. The method of claim 3, wherein the ultrasonic cleaning by using the organic solvent is conducted at a frequency of 15 KHz to 25 KHz.

5. The method of claim 3, wherein the ultrasonic cleaning by using heated deionized water is conducted at a frequency of 20 KHz to 35 KHz; and the ultrasonic cleaning by using heated deionized water is conducted at a temperature of 50° C. to 80° C.

6. The method of claim 1, wherein the partial dezincification is conducted by a process comprising: mixing a resulting pretreated copper-zinc alloy with an acid solution to obtain a mixture; subjecting the mixture to replacement to obtain a reaction system; and subjecting the reaction system to washing with a weakly alkaline solution, washing with water, and drying in sequence.

7. The method of claim 6, wherein the acid solution is one selected from the group consisting of a nitric acid solution, a sulfuric acid solution, and a hydrochloric acid solution.

8. A copper-zinc alloy catalyst prepared by the method of claim 1.

9. A method for preparing a low-carbon alcohol from a synthesis gas by using the copper-zinc alloy catalyst of claim 8, comprising:
subjecting the copper-zinc alloy catalyst to impurity removal to obtain a cleaned copper-zinc alloy, introducing the synthesis gas into the cleaned copper-zinc alloy, and subjecting the synthesis gas to reaction at a temperature of 240° C. to 340° C. to obtain the low-carbon alcohol; wherein the synthesis gas is introduced at a pressure of 3 MPa to 5 MPa.

10. The method of claim 9, wherein the synthesis gas is introduced at a flow rate of 80 mL/min to 120 mL/min and a volume space velocity of 9,600 h⁻¹ to 14,400 h⁻¹; the synthesis gas consists of H₂ and CO; and a volume ratio of the H₂ to the CO is in a range of (1-3):1.

11. The copper-zinc alloy catalyst of claim 8, wherein the copper-zinc alloy particles are prepared from brass.
